(19) United States
(12) Patent Application Publication
(10) Pub. No.: US 2013/0289657 A1
(43) Pub. Date: Oct. 31, 2013

(54) PROGRAMMER UPGRADING METHODS, IMPLANTABLE MEDICAL DEVICE PROGRAMMERS, AND ARTICLES OF MANUFACTURE

(71) Applicant: Alberto A. RODRIGUEZ, Friendswood, TX (US)

(72) Inventor: Alberto A. RODRIGUEZ, Friendswood, TX (US)

(73) Assignee: CYBERONICS, INC., Houston, TX (US)

(21) Appl. No.: 13/455,376

(22) Filed: Apr. 25, 2012

(51) Int. Cl. A61N 1/08

(52) U.S. Cl. 607/59

(57) ABSTRACT

A method includes receiving identification data at a medical device programmer from a personal medical device via an electronic communication link between the medical device programmer and the personal medical device. The method includes receiving missing programming code at the medical device programmer, via the electronic communication link, from the personal medical device identified in the identification data. The medical device programmer is upgraded using the received missing programming code. The upgraded medical device programmer includes the missing programming code configured to program the identified personal medical device. The medical device programmer may be an IMD programmer including processing circuitry configured to receive identification data from an IMD. The processing circuitry is also configured to receive a missing programming module from the identified IMD for a module manager of the IMD programmer.

### TECHNICAL FIELD

The embodiments pertain to methods that include upgrading medical device programmers, implantable medical device (IMD) programmers, and articles of manufacture storing programming configured to upgrade IMD programmers.

### BACKGROUND

A variety of known medical devices include electronic components. For some devices, such as an implantable medical device (IMD), a medical device programmer may be used to program settings for the IMD, interrogate the IMD to collect data, diagnose malfunctions, and otherwise communicate therewith. Historically, IMDs suffered from limited memory storage capacity, limited communication bandwidth creating a data transfer hindrance between the medical device programmer and the IMD, and limited power supply capacity. Storage, bandwidth, and power limitations led to limited software capabilities.

Health care personnel that utilize such medical device programmers may upgrade a medical device programmer to accommodate new medical devices. For IMDs, software upgrades to IMD programmers are often distributed on compact flash memory cards or other memory devices by a manufacturer's representative to health care facilities where the contents of the memory card are installed on the IMD programmer to upgrade the IMD programmer software. When a patient visits a health care facility that uses a medical device programmer not yet upgraded to accommodate a new medical device, health care personnel must obtain the software upgrade prior to servicing the medical device.

Changes may be implemented in medical devices and methods for upgrading medical device programmers to overcome the described limitations.

### SUMMARY

An IMD programmer includes a wireless electronic communication device and processing circuitry. The processing circuitry is configured to receive identification data from an IMD via the wireless electronic communication device. The identification data identifies the IMD. The processing circuitry is also configured to receive a missing programming module via the wireless electronic communication device from the identified IMD. The processing circuitry is further configured to upgrade the IMD programmer by including the received programming module in a module manager of the IMD programmer. The upgraded IMD programmer includes the programming module configured to program the identified IMD.

A method includes receiving identification data at a medical device programmer from a personal medical device, such as an IMD, via an electronic communication link between the medical device programmer and the personal medical device. The method includes receiving missing programming code at the medical device programmer, via the electronic communication link, from the personal medical device identified in the identification data. The medical device programmer is upgraded using the received missing programming code. The upgraded medical device programmer includes the missing programming code configured to program the identified personal medical device.

An article of manufacture includes a computer-readable storage medium storing programming configured to cause processing circuitry to perform processing that includes receiving identification data from an IMD via a wireless communication link. The identification data identifies the IMD. The processing also includes receiving a missing programming module, via the wireless communication link, from the identified IMD. The processing further includes upgrading the article of manufacture by including the received programming module in a module manager of the article of manufacture. The upgraded article of manufacture includes the programming module configured to program the identified IMD.

### BRIEF DESCRIPTION OF THE DRAWINGS

Some embodiments of the invention are described below with reference to the following accompanying drawings.

FIGS. 1 and 3 are block diagrams of implantable medical device (IMD) programmers according to two embodiments.

FIG. 2 is a block diagram of a system according to an embodiment including the IMD programmer of FIG. 1 in communication with an IMD.

FIG. 4 is a block diagram of an IMD according to a further embodiment.

FIGS. 5 and 7 are flow diagrams of methods according to two embodiments that include upgrading a medical device programmer.

FIG. 6 is a flow diagram of a method that may be included in the embodiments herein.

### DETAILED DESCRIPTION

Recent advances and expected future advances in the technology of medical devices, such as implantable medical devices (IMDs), promise to enable new functionalities for next generation medical devices. For example, increases in storage capacity of memory components, increases in communication bandwidth, and increases in power supply capacity may individually, or in combination, enable the distributions of upgrades for medical device programmers using the medical devices themselves. Programmer upgrades may be stored in a medical device and transferred to a programmer, when missing from the programmer, to allow a programmer upgrade.

Software and hardware constraints of IMDs and IMD programmers may benefit from further modification. For example, known software for IMD programmers relies on a monolithic architecture that includes all information used to communicate with all models of a manufacturer's IMDs. Accordingly, upgrading the software of an IMD programmer may involve installing the entire monolithic architecture. Hence, the distribution of software upgrades having monolithic architectures may require a significant amount of memory. A modular architecture may be used by the IMD programmers to reduce the amount of memory required to distribute the software upgrades, thereby reducing the memory burden on the IMD when the IMD is used as a distribution mechanism.

In an embodiment, an IMD programmer includes a wireless electronic communication device and processing circuitry. The processing circuitry is configured to receive identification data from an IMD via the wireless electronic communication device. The identification data identifies the IMD. The processing circuitry is also configured to receive a missing programming module via the wireless electronic communication device from the identified IMD. The processing circuitry is further configured to upgrade the IMD programmer by including the received programming module in a module manager of the IMD programmer. The upgraded IMD programmer includes the programming module configured to program the identified IMD.

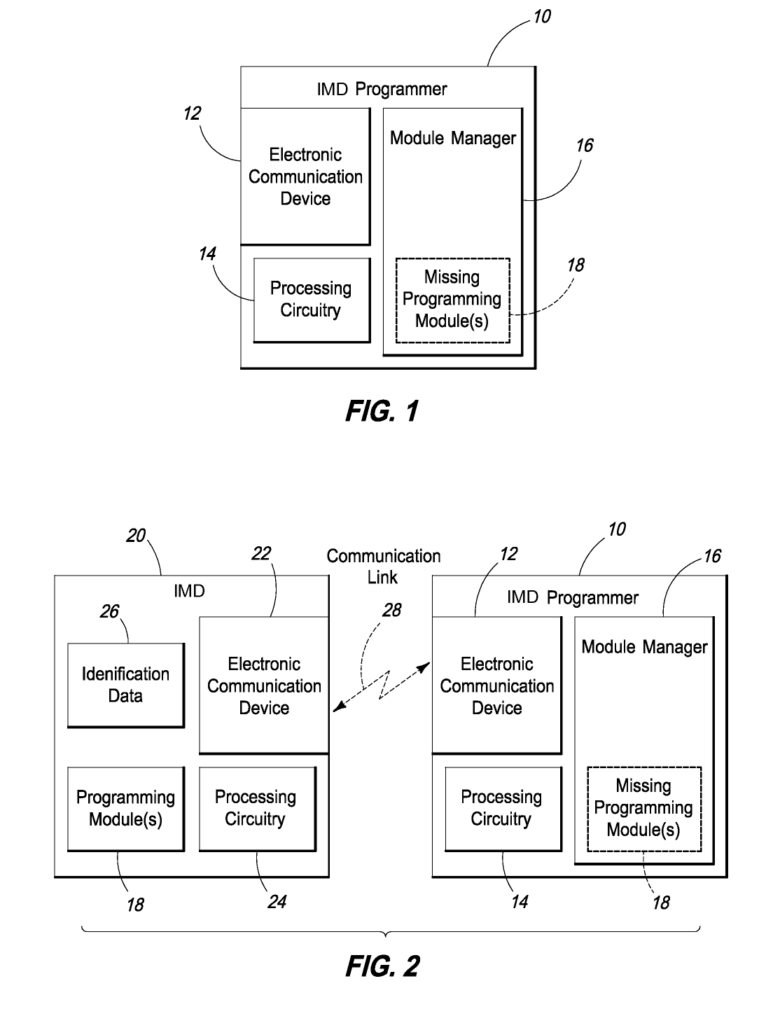

FIG. 1 shows a diagram of an IMD programmer 10 that includes an electronic communication device 12. Electronic communication device 12 may be a wireless communication device. IMD programmer 10 further includes processing circuitry 14 and a module manager 16. Notably, FIG. 1 represents a missing programming module 18 using dashed lines. For IMD programmer 10, processing circuitry 14 may be configured to receive identification data from an IMD (not shown), receive missing programming module 18 from the identified IMD, and upgrade IMD programmer 10. Missing programming module 18 received from the IMD may be provided to module manager 16. Thereafter, upgraded IMD programmer 10 may be configured to program the identified IMD.

The IMD programmer of the described embodiments represents a method and device for upgrading IMD programmers. The IMD may be used itself to upgrade an IMD programmer incapable at the outset to program the IMD. Embodiments may also encompass upgrading an IMD programmer even though it is capable of programming the IMD in the circumstance where the IMD carries updated programming code. Further embodiments include an IMD programmer having a monolithic architecture in which the monolithic software upgrade is provided by the IMD to the IMD programmer.

By way of example, the wireless communication device of the IMD programmer may include a hand-held wand having an antenna configured for placement in proximity to an IMD. For example, the hand-held wand may be placed in contact or nearly in contact with a patient's clothing and/or skin overlying the IMD. The closeness in proximity of the hand-held wand to the IMD may assist in reducing unwanted wireless interaction with the IMD. The wand may be physically connected to the IMD programmer with a cable or communicate by another means. In some embodiments, the IMD programmer may be a computing device, such as a personal digital assistant (PDA), a mobile device (e.g., a smart phone), a tablet, a laptop, or any other mobile or stationary computing device. The wireless communication device of the IMD programmer may be configured to communicate with the IMD in one or more of the ISM (Industrial, Scientific, and Medical) bands, in the MICS (Medical Implant Communication Service) band, an unrestricted band, in any other appropriate frequency band, or at any other appropriate frequency. In some embodiments, the wireless communication may be accomplished using propagating electromagnetic waves (far-field communication) or a non-propagating magnetic field (near-field communication).

The processing circuitry may be configured to send IMD interrogation data via the wireless electronic communication device before receiving the identification data. The processing circuitry may be configured to send a request for the missing programming module to the identified IMD. The processing circuitry may be configured to determine that the IMD programmer lacks the programming module (or software upgrade) configured to program the identified IMD. Accordingly, the module manager and/or other programming code of the IMD programmer may exhibit some minimal level of communication ability with any model of an IMD with which the IMD programmer is designed to communicate to identify the IMD and receive the missing programming module (or software upgrade) to upgrade the IMD programmer.

A system may include the described IMD programmer and an IMD. The IMD may include an IMD electronic communication device and IMD processing circuitry configured to send the IMD identification data via the IMD electronic communication device to the IMD programmer. The IMD processing circuitry may also be configured to send the missing programming module (or software upgrade) via the IMD electronic communication device to the IMD programmer. The IMD processing circuitry may be configured to determine that the IMD programmer lacks the programming module (or software upgrade) configured to program the identified IMD. Such an option may be alternative to the option described above wherein the processing circuitry of the IMD programmer is configured to determine that the IMD programmer lacks the programming module (or software upgrade). It is further conceivable that the two options may be used in combination. The IMD may further include a computer readable storage medium in which the programming module (or software upgrade) is stored. Processing circuitry described herein, whether for the IMD or the IMD programmer may be configured to provide the described features by software, hardware, or a combination thereof.

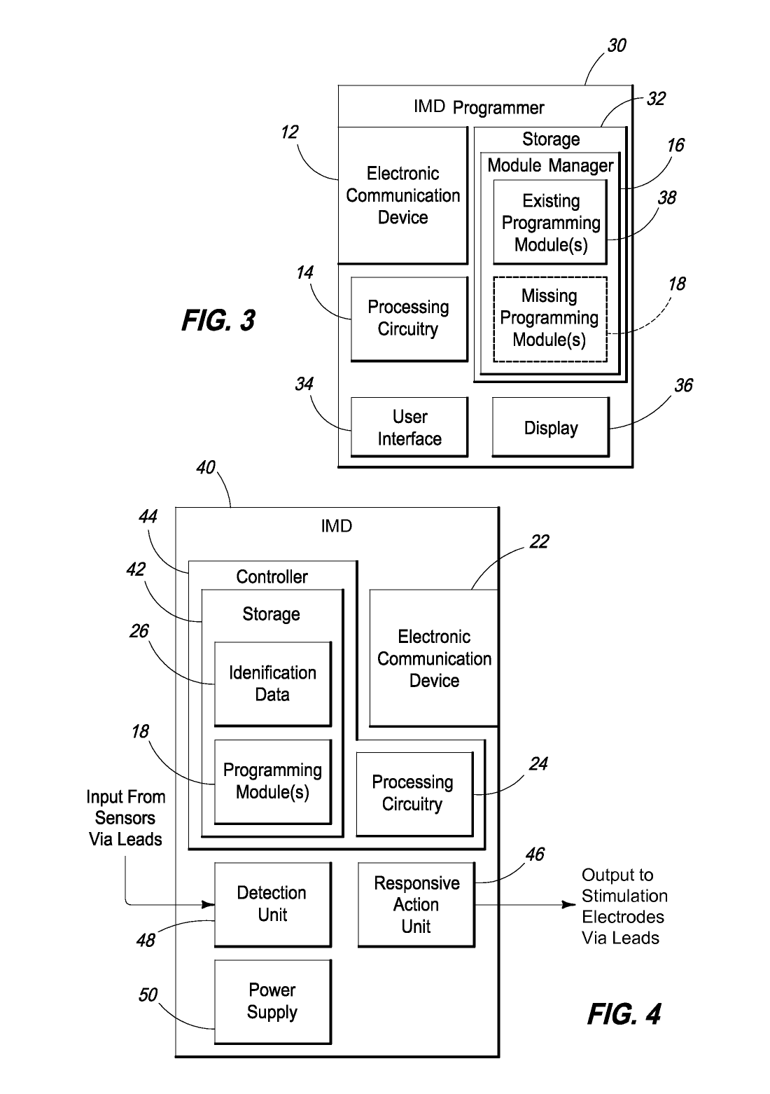

FIG. 2 shows a diagram that includes IMD programmer 10 of FIG. 1 in a system that includes an IMD 20 with an electronic communication device 22 and processing circuitry 24. A communication link 28 exists between IMD 20 and IMD programmer 10 using respective electronic communication device 22 and electronic communication device 12. IMD 20 further includes identification data 26 and programming module 18. Notably, programming module 18, although represented in FIGS. 1 and 2 in the singular, may be plural. That is, it is conceivable that more than one programming module received from the identified IMD may be used to upgrade the IMD programmer. In some embodiments, the IMD programmer 10 may have a monolithic architecture in which the monolithic software upgrade is provided by the IMD to the IMD programmer.

Identification data 26 of IMD 20 may allow IMD 20 to be self-sustaining In other words, IMD 20 may possess the ability of itself to communicate to IMD programmer 10 sufficient information so that IMD programmer 10 may determine whether programming module 18 associated with programming of IMD 20 is missing. As the term is used herein, the ability of a medical device programmer to “program” a medical device refers to interrogation of the device to acquire characteristic information and/or other data, modification of device settings, diagnosis of device malfunction, etc.

Although a number of benefits exist for the described IMD programmer, IMD, and system combining the two, forward compatibility bears great significance. Certainly, the ability of the module manager to obtain a missing programming module from the identified IMD provides a level of forward compatibility. In some embodiments, the IMD programmers with a monolithic architecture may require an upgrade of the entire architecture to provide compatibility with new devices as they become available. It is also conceivable that even the module manager of the module architecture may warrant upgrade at some point.

At that time, the forward compatibility features of the module manager provide a further benefit. Specifically, the module manager may be upgraded perhaps without regard to the accumulated modules for various IMDs previously serviced by the IMD programmer. Although the accumulated programming modules might be retained, the IMD programmer could also continue without them, relying instead on obtaining programming modules from further IMDs as they are serviced in the future by the IMD programmer using the upgraded module manager.

FIG. 3 shows a diagram of an IMD programmer 30 that includes electronic communication device 12, processing circuitry 14, and module manager 16 of FIG. 1. Programming module 18 is represented as missing from IMD programmer 30 using dashed lines. Module manager 16 may also include existing programming modules 38 that may be used for programming associated IMDs either because existing programming modules 38 were included originally with module manager 16 upon installation, were obtained from an IMD, or were obtained from other sources, such as internet downloading from a manufacturer's product support.

IMD programmer 30 includes computer readable storage 32, which contains module manager 16 and existing programming modules 38. Programming module 18 is absent from storage 32. Storage 32 may include any of a variety of computer readable storage media, depending on the specific hardware in which IMD programmer 30 is implemented. As one example, IMD programmer may be implemented in a general-purpose computer, though other implementations are conceivable. IMD programmer 30 may also include a user interface 34 and a display 36. User interface 34 and display 36 may enable user control of IMD programmer 30 for programming of an IMD, including interrogation, settings control, malfunction diagnosis, etc.

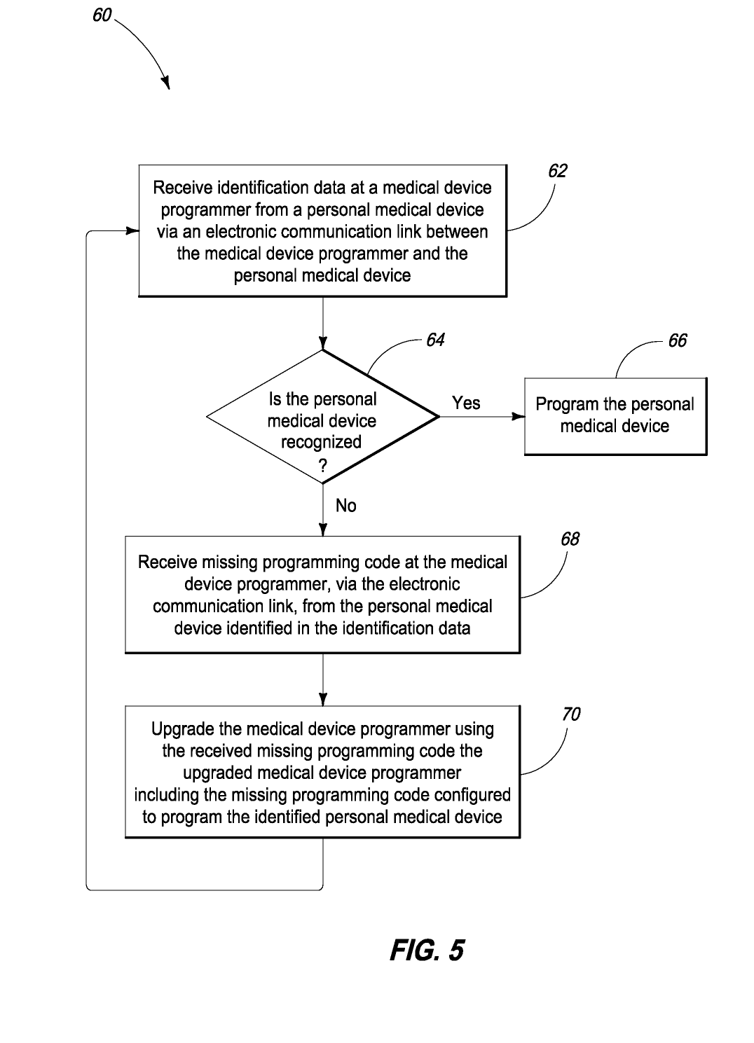

FIG. 4 shows an IMD 40 that includes electronic communication device 22, processing circuitry 24, and identification data 26 of the IMD shown in FIG. 2. IMD 40 additionally includes a controller 44 controlling the operation of computer readable storage 42 and processing circuitry 24. Storage 42 in turn includes identification data 26 and programming module 18. Processing circuitry 24 of controller 44 may further be configured to operate a responsive action unit 46 and a detection unit 48 relying on a power supply 50.

While embodiments described herein may be implemented in a variety of medical devices, IMD 40 allows output to stimulation electrodes via leads from responsive action unit 46. IMD 40 also allows input from sensors via leads to detection unit 48. Responsive action unit 46 may include hardware and/or firmware to initiate one or more of responsive actions such as alarms, warnings, health-related event determinations, logging/recording information related to the health event, or therapies such as electrical stimulation applied to a target tissue via leads shown in FIG. 4. Controller 44 may be generally operable to control various aspects of IMD 40. IMD 40 may receive body data signals from sensors via leads into detection unit 48 for processing by controller 44. Detection unit 48 may detect a variety of health-related events, such as a seizure event. Responsive action unit 46 may then provide therapies, such as electrical pulses to modulate the seizure event.

As may be appreciated from the discussion herein regarding various medical devices, such as IMD programmers, IMDs, and systems combining the two, embodiments also include a variety of methods related to operating such devices. One method includes receiving identification data at a medical device programmer from a personal medical device via an electronic communication link between the medical device programmer and the personal medical device. The method includes receiving missing programming code at the medical device programmer, via the electronic communication link, from the personal medical device identified in the identification data. The medical device programmer is upgraded using the received missing programming code. The upgraded medical device programmer includes the missing programming code configured to program the identified personal medical device.

By way of example, the personal medical device may include an implantable pulse generator. The method may further include sending interrogation data from a medical device programmer to the personal medical device via the electronic communication link before receiving the identification data. In keeping with other descriptions herein, the electronic communication link may be wireless. The electronic communication link may be established by the medical device programmer interrogating the personal medical device, wherein the personal medical device is an implantable medical device. Medical devices other than implantable pulse generators are contemplated. The method may further include sending a request for the missing programming code from the medical device programmer to the identified personal medical device.

As a further example, the missing programming code in the described method may be a programming module. As such, the medical device programmer may include a module manager. The upgrading described may comprise including the programming module in the module manager. The medical device programmer may lack at least one programming module configured to program the personal medical device prior to upgrading the medical device programmer. Alternatively, the medical device programmer may include an outdated version of the programming module, but lack an updated version prior to upgrading the medical device programmer. The received programming module may include the updated version of the programming module.

The method may further include determining at the medical device programmer that the medical device programmer lacks the programming code based on the identification data. As an alternative, the medical device itself may determine that the medical device programmer lacks the programming code. Receipt of the missing programming code from the personal medical device may occur after a failed attempt by the medical device programmer to obtain the missing programming code via the Internet.

It will be appreciated that a manufacturer's customer support website may present a convenient resource for obtaining missing programming code, such as programming modules. Although access to the Internet is known for being somewhat ubiquitous, reliance on Internet access may be problematic in the context of health care facilities. Consequently, methods described herein for obtaining missing programming code from a personal medical device or obtaining missing programming modules from an IMD may constitute a primary means for obtaining the code/module or a secondary means after primarily relying on the Internet. A setting, such as an operating room, may present a circumstance in which Internet access is less likely, but timely access to missing programming codes/modules is desired.

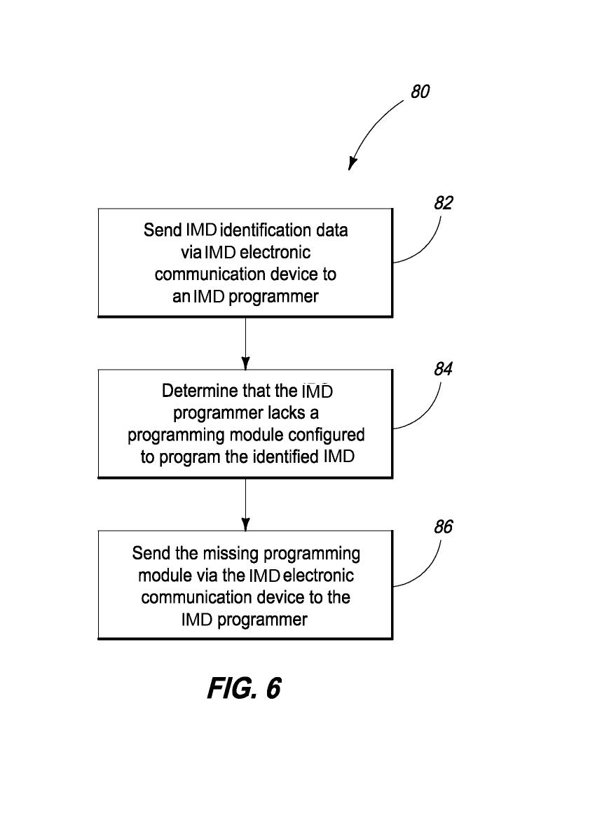

FIG. 5 shows a flow diagram of a method 60 that includes a step 62 of receiving identification data at a medical device programmer from a personal medical device. An inquiry is made in step 64 regarding whether the personal medical device is recognized. If it is, the personal medical device may be programmed in step 66. If it is not, method 60 includes receiving missing programming code at the medical device programmer in step 68. Step 70 then includes upgrading the medical device programmer using the received missing programming code. After the medical device programmer is upgraded with the missing programming code, the medical device programmer may then program the personal medical device.

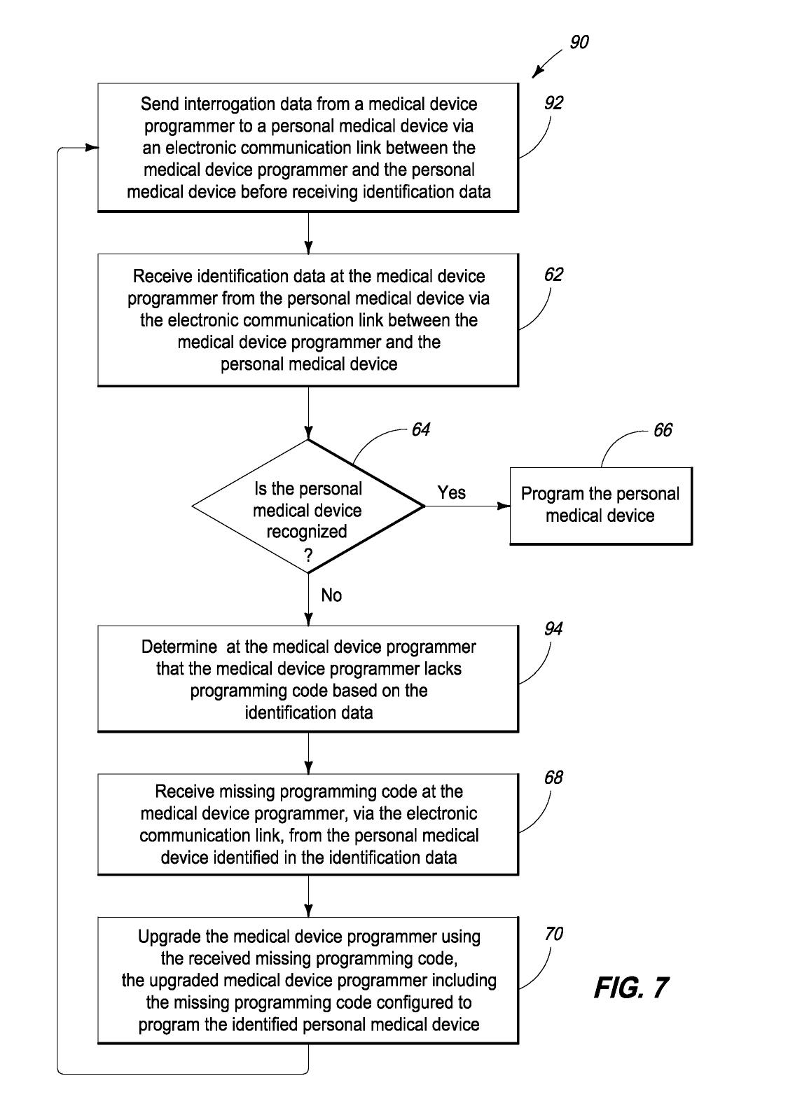

FIG. 6 shows a flow diagram of a method 80 that may be implemented in an IMD. It will be appreciated that implementations in other medical devices are conceivable. Step 82 includes sending IMD identification data via an IMD electronic communication device to an IMD programmer. Method 80 includes in step 84 determining that the IMD programmer lacks a programming module configured to program the identified IMD. In step 86 the missing programming module is sent via the IMD electronic communication device to the IMD programmer.

FIG. 7 shows a flow diagram of a method 90 including steps 62, 64, 66, 68, and 70 of method 60 in FIG. 5 along with additional steps. In step 92 interrogation data is sent from a medical device programmer to a personal medical device via an electronic communication link between the medical device programmer and the personal medical device before receiving identification data. Step 62 includes receiving identification data at the medical device programmer from the personal medical device. Thereafter, if the personal medical device is recognized in step 64, the personal medical device may be programmed in step 66. Otherwise, method 90 includes determination in step 94 at the medical device programmer that the medical device programmer lacks programming code based on the identification data.

As in method 60, method 90 then includes receiving missing programming code at the medical device programmer in step 68. Next, step 70 includes upgrading the medical device programmer using the received missing programming code. Thereafter, method 90 returns to step 92 to program the personal medical device, assuming a successful upgrade in the preceding steps.

As mentioned, embodiments herein may include obtaining programming codes/modules through the Internet and other interacting with product support. In the context of method 60 shown in FIG. 5, following failure in step 64 to recognize the personal medical device, a connection may be established through Internet communication with a manufacturer's product support. The missing programming code may be requested and downloaded. If no connection to product support may be established or the download fails, step 68 may proceed with receiving missing programming code from the personal medical device.

Additional interaction with product support is contemplated. For example, in the event of a successful download of programming code, the manufacturer's product support may be notified of a new installation. Thereafter, a tutorial of the personal medical device associated with the downloaded programming code may be launched from product support.

In an embodiment, an article of manufacture includes a computer-readable storage medium storing programming configured to cause processing circuitry to perform processing that includes receiving identification data from an IMD via a wireless communication link. The identification data identifies the IMD. The processing also includes receiving a missing programming module, via the wireless communication link, from the identified IMD. The processing further includes upgrading the article of manufacture by including the received programming module in a module manager of the article of manufacture. The upgraded article of manufacture includes the programming module configured to program the identified IMD.

By way of example, the processing may further include determining that the article of manufacture lacks the programming module configured to program the identified IMD. Examples of computer-readable media include any one of electronic, magnetic, optical, electromagnetic, infrared, or semiconductor media. Some specific examples of computer-readable media include, but are not limited to magnetic computer diskettes, hard drives, random access memory, read only memory, flash memory, and other configurations capable of storing programming, data, or other digital information.

Benefits of the embodiments are described herein but, in summary, include reducing the use of memory cards, such as flash media or other software distribution media, to upgrade medical device programmers to accommodate new devices. Consequently, the embodiments may reduce site visits by manufacturer's personnel to perform such upgrades. Even so, the embodiments increase the likelihood of health care personnel having an upgraded medical device programmer with forward compatibility to a new medical device. Given such benefits, distribution of programming code to the field may be expedited. Even though next generation IMDs may possess increased storage capacity, increased communication bandwidth, and increased power supply, the concept of modularized architecture allows manageably sized data files to be transferred containing missing programming code. The manageable size reduces storage requirements, demand on communication and electronics, and power usage of finite power supplies in implantable devices.

In compliance with the statute, the embodiments have been described in language more or less specific as to structural and methodical features. It is to be understood, however, that the embodiments are not limited to the specific features shown and described. The embodiments are, therefore, claimed in any of their forms or modifications within the proper scope of the appended claims appropriately interpreted in accordance with the doctrine of equivalents.

What is claimed is:

1. A method comprising:
receiving identification data at a medical device programmer from a personal medical device via an electronic communication link between the medical device programmer and the personal medical device;
receiving missing programming code at the medical device programmer, via the electronic communication link, from the personal medical device identified in the identification data; and
upgrading the medical device programmer using the received missing programming code, the upgraded medical device programmer including the missing programming code configured to program the identified personal medical device.

2. The method of claim 1, wherein the personal medical device comprises an implantable medical device.

3. The method of claim 1, further comprising sending interrogation data from the medical device programmer to the personal medical device via the electronic communication link before receiving the identification data.

4. The method of claim 1, wherein the electronic communication link is wireless and is established by the medical device programmer interrogating the personal medical device, wherein the personal medical device is an implantable medical device.

5. The method of claim 1, further comprising sending a request for the missing programming code from the medical device programmer to the identified personal medical device.

6. The method of claim 1, wherein the missing programming code is a programming module, the medical device programmer comprises a module manager, and the upgrading comprises including the programming module in the module manager.

7. The method of claim 6, wherein the medical device programmer lacks at least one programming module configured to program the personal medical device prior to upgrading the medical device programmer.

8. The method of claim 6, wherein the medical device programmer includes an outdated version of the programming module, but lacks an updated version prior to upgrading the medical device programmer, and the received programming module includes the updated version of the programming module.

9. The method of claim 1, further comprising determining at the medical device programmer that the medical device programmer lacks the programming code based on the identification data.

10. The method of claim 1, wherein the receiving missing programming code from the personal medical device occurs after a failed attempt by the medical device programmer to obtain the missing programming code via the Internet.

11. An implantable medical device (IMD) programmer comprising a wireless electronic communication device and processing circuitry, the processing circuitry configured to:
receive identification data from an IMD via the wireless electronic communication device, the identification data identifying the IMD;
receive a missing programming module via the wireless electronic communication device from the identified IMD; and
upgrade the IMD programmer by including the received programming module in a module manager of the IMD programmer, the upgraded IMD programmer including the programming module configured to program the identified IMD.

12. The programmer of claim 11, wherein the wireless communication device comprises a hand-held wand having an antenna configured to be placed in proximity to an IMD.

13. The programmer of claim 11, wherein the processing circuitry is configured to send IMD interrogation data via the wireless electronic communication device before receiving the identification data.

14. The programmer of claim 11, wherein the processing circuitry is configured to send a request for the missing programming module to the identified IMD.

15. The programmer of claim 11, wherein the processing circuitry is configured to determine that the IMD programmer lacks the programming module configured to program the identified IMD.

16. A system comprising the IMD programmer of claim 11 and an IMD, the IMD including an IMD electronic communication device and IMD processing circuitry configured to:
send the IMD identification data via the IMD electronic communication device to the IMD programmer; and
send the missing programming module via the IMD electronic communication device to the IMD programmer.

17. The system of claim 16, wherein the IMD processing circuitry is configured to determine that the IMD programmer lacks the programming module configured to program the identified IMD.

18. The system of claim 16, wherein the IMD further comprises a computer-readable storage medium in which the programming module is stored.

19. An article of manufacture comprising a computer-readable storage medium storing programming configured to cause processing circuitry to perform processing comprising:
receiving identification data from an implantable medical device (IMD) via a wireless communication link, the identification data identifying the IMD;
receiving a missing programming module, via the wireless communication link, from the identified IMD; and
upgrading the article of manufacture by including the received programming module in a module manager of the article of manufacture, the upgraded article of manufacture including the programming module configured to program the identified IMD.

20. The article of manufacture of claim 19, wherein the processing further comprises determining that the article of manufacture lacks the programming module configured to program the identified IMD.
